SEQUENCE LISTING (50 sequences)

SEQ ID NO: 1 - protein, 4 aa, Artificial Sequence
  Synthetic
RGDS

SEQ ID NO: 2 - protein, 5 aa, Artificial Sequence
  Synthetic
CRGDS

SEQ ID NO: 3 - protein, 6 aa, Artificial Sequence
  Synthetic
CRGDSP

SEQ ID NO: 4 - protein, 5 aa, Artificial Sequence
  Synthetic
PHSRN

SEQ ID NO: 5 - protein, 9 aa, Artificial Sequence
  Synthetic
GWGGRGDSP

SEQ ID NO: 6 - protein, 13 aa, Artificial Sequence
  Synthetic
SIDQVEPYSSTAQ

SEQ ID NO: 7 - protein, 11 aa, Artificial Sequence
  Synthetic
GRNIAEIIKDI

SEQ ID NO: 8 - protein, 9 aa, Artificial Sequence
  Synthetic
DITYVRLKF

SEQ ID NO: 9 - protein, 9 aa, Artificial Sequence
  Synthetic
DITVTLNRL

SEQ ID NO: 10 - protein, 8 aa, Artificial Sequence
  Synthetic
GRYVVLPR

SEQ ID NO: 11 - protein, 13 aa, Artificial Sequence
  Synthetic
GNRWHSIYITRFG

SEQ ID NO: 12 - protein, 12 aa, Artificial Sequence
  Synthetic
GASIKVAVSADR

SEQ ID NO: 13 - protein, 9 aa, Artificial Sequence
  Synthetic
GTTVKYIFR

SEQ ID NO: 14 - protein, 10 aa, Artificial Sequence
  Synthetic
GSIKIRGTYS

SEQ ID NO: 15 - protein, 7 aa, Artificial Sequence
  Synthetic
GSINNNR

SEQ ID NO: 16 - protein, 9 aa, Artificial Sequence
  Synthetic
SDPGYIGSR

SEQ ID NO: 17 - protein, 5 aa, Artificial Sequence
  Synthetic
YIGSR

SEQ ID NO: 18 - protein, 14 aa, Artificial Sequence
  Synthetic
GTPGPQGIAGQGVV

SEQ ID NO: 19 - protein, 14 aa, Artificial Sequence
  Synthetic
GTPGPQGIAGQRVV

SEQ ID NO: 20 - protein, 7 aa, Artificial Sequence
  Synthetic
MNYYSNS

SEQ ID NO: 21 - protein, 12 aa, Artificial Sequence
  Synthetic
KKQRFRHRNRKG

SEQ ID NO: 22 - protein, 18 aa, Artificial Sequence
  Synthetic
GGGKLTWQELYQLKYKGI

SEQ ID NO: 23 - protein, 15 aa, Artificial Sequence
  Synthetic
KLTWQELYQLKYKGI

SEQ ID NO: 24 - protein, 12 aa, Artificial Sequence
  Synthetic
KIPKASSVPTEL

SEQ ID NO: 25 - protein, 20 aa, Artificial Sequence
  Synthetic
KIPKASSVPTELSAISTLYL

SEQ ID NO: 26 - protein, 8 aa, Artificial Sequence
  Synthetic
KRTGQYKL

SEQ ID NO: 27 - protein, 14 aa, Artificial Sequence
  Synthetic
KCGGPQGIWGQGCK

SEQ ID NO: 28 - protein, 14 aa, Artificial Sequence
  Synthetic
KCGGPQGIAGQGCK

SEQ ID NO: 29 - protein, 22 aa, Artificial Sequence
  Synthetic
CRGDGGGGGGGGGGGGGPHSRN

SEQ ID NO: 30 - protein, 19 aa, Artificial Sequence
  Synthetic
CPHSRNSGSGSGSGSGRGD

SEQ ID NO: 31 - protein, 10 aa, Artificial Sequence
  Synthetic
GCYGRGDSPG

SEQ ID NO: 32 - protein, 5 aa, Artificial Sequence
  Synthetic
CRDGS

SEQ ID NO: 33 - protein, 5 aa, Artificial Sequence
  Synthetic
  MISC_FEATURE (4)..(4): D-phenylalanine
RGDFC

SEQ ID NO: 34 - protein, 17 aa, Artificial Sequence
  Synthetic
  MISC_FEATURE (3)..(3): D-phenylalanine
  MISC_FEATURE (4)..(4): D-alanine
  MISC_FEATURE (5)..(5): D-tyrosine
  MISC_FEATURE (6)..(6): D-leucine
CEFAYLIDFNWEYPASK

SEQ ID NO: 35 - protein, 18 aa, Artificial Sequence
  Synthetic
  MISC_FEATURE (3)..(3): D-alanine
  MISC_FEATURE (7)..(7): D-phenylalanine
  MISC_FEATURE (13)..(13): D-tyrosine
  MISC_FEATURE (17)..(17): D-leucine
CDAPYNFEFAWEYVISLK

SEQ ID NO: 36 - protein, 12 aa, Artificial Sequence
  Synthetic
RKRLQVQLSIRT

SEQ ID NO: 37 - protein, 5 aa, Artificial Sequence
  Synthetic
IKVAV

SEQ ID NO: 38 - protein, 5 aa, Artificial Sequence
  Synthetic
YIGSR

SEQ ID NO: 39 - protein, 8 aa, Artificial Sequence
  Synthetic
KRTGQYKL

SEQ ID NO: 40 - protein, 7 aa, Artificial Sequence
  Synthetic
TYRSRKY

SEQ ID NO: 41 - protein, 17 aa, Artificial Sequence
  Synthetic
KRTGQYKLGSKTGPGQK

SEQ ID NO: 42 - protein, 14 aa, Artificial Sequence
  Synthetic
QAKHKQRKRLKSSC

SEQ ID NO: 43 - protein, 17 aa, Artificial Sequence
  Synthetic
SPKHHSQRARKKKNKNC

SEQ ID NO: 44 - protein, 6 aa, Artificial Sequence
  Synthetic
  MISC_FEATURE (1)..(1): X is a hydropathic residue
  MISC_FEATURE (2)..(2): B is a basic residue
  MISC_FEATURE (3)..(3): B is a basic residue
  MISC_FEATURE (4)..(4): X is a hydropathic residue
  MISC_FEATURE (5)..(5): B is a basic residue
  MISC_FEATURE (6)..(6): X is a hydropathic residue
XBBXBX

SEQ ID NO: 45 - protein, 8 aa, Artificial Sequence
  Synthetic
  MISC_FEATURE (1)..(1): X is a hydropathic residue
  MISC_FEATURE (2)..(2): B is a basic residue
  MISC_FEATURE (3)..(3): B is a basic residue
  MISC_FEATURE (4)..(4): B is a basic residue
  MISC_FEATURE (5)..(5): X is a hydropathic residue
  MISC_FEATURE (6)..(6): X is a hydropathic residue
  MISC_FEATURE (7)..(7): B is a basic residue
  MISC_FEATURE (8)..(8): X is a hydropathic residue
XBBBXXBX

SEQ ID NO: 46 - protein, 5 aa, Artificial Sequence
  Synthetic
RGDSP

SEQ ID NO: 47 - protein, 6 aa, Artificial Sequence
  Synthetic
CRGDGS

SEQ ID NO: 48 - protein, 6 aa, Artificial Sequence
  Synthetic
CIKVAV

SEQ ID NO: 49 - protein, 14 aa, Artificial Sequence
  Synthetic
KCGGPQGIWGQGCK

SEQ ID NO: 50 - protein, 14 aa, Artificial Sequence
  Synthetic
KCGGPQGIAGQGCK

(19) United States
(12) Patent Application Publication
(10) Pub. No.: US 2020/0017823 A1
(43) Pub. Date: Jan. 16, 2020

(54) HYDROGEL COMPOSITIONS FOR USE IN NEURAL CELL EXPANSION AND DIFFERENTIATION

(71) Applicant: Wisconsin Alumni Research Foundation, Madison, WI (US)

(72) Inventors: William Leo Murphy, Waunakee, WI (US); William Timothy Daly, Madison, WI (US); Jonathan Lloyd Evans, Madison, WI (US); Elizabeth Anne Aisenbrey, Madison, WI (US); Elizabeth Emma Torr, Madison, WI (US)

(21) Appl. No.: 16/510,469

(22) Filed: Jul. 12, 2019

Related U.S. Application Data
(60) Provisional application No. 62/697,646, filed on Jul. 13, 2018.

(51) Int. Cl. C12N 5/00; C12N 5/0793; G01N 33/50

(52) U.S. Cl. CPC C12N 5/0018; C12N 5/0619; C12N 2501/999; C12N 2500/50; C12N 2501/998; G01N 33/5014

(57) ABSTRACT

Hydrogel compositions and methods of using hydrogel compositions are disclosed. Advantageously, the hydrogel compositions offer the ability to promote cellular expansion and/or cellular differentiation of various neuronal cells. The hydrogel compositions can further be used in toxicity screening assays for neurotoxicants.

### CROSS-REFERENCE TO RELATED APPLICATION

This application claims priority to U.S. Provisional Application Ser. No. 62/697,646, filed on Jul. 13, 2018, which is hereby incorporated by reference in its entirety.

### STATEMENT REGARDING FEDERALLY SPONSORED RESEARCH OR DEVELOPMENT

This invention was made with government support under TR000506 awarded by the National Institutes of Health and 83573701 awarded by the Environmental Protection Agency. The government has certain rights in the invention.

### STATEMENT IN SUPPORT FOR FILING A SEQUENCE LISTING

A paper copy of the Sequence Listing and a computer readable form of the Sequence Listing containing the file named “P160311US02_ST25.txt”, which is 12,029 bytes in size (as measured in MICROSOFT WINDOWS® EXPLORER), are provided herein and are herein incorporated by reference. This Sequence Listing consists of SEQ ID NOs:1-50.

### BACKGROUND OF THE DISCLOSURE

The present disclosure relates generally to methods for preparing biomaterial compositions and methods for using the biomaterial compositions. More particularly, the present disclosure relates to hydrogel compositions and to methods for using the hydrogel compositions to promote neural cell expansion and neural cell differentiation.

Throughout the process of neural development, including from their earliest neuronal precursor to terminally differentiated neurons surrounded by their supporting glial cells, there is a need to have defined control over key aspects of the neurodevelopment process. It has been demonstrated that neurons, glia cells (e.g. astrocytes) and the like have different interactions with their surrounding environment based on their location within the central nervous system (CNS). For example, a neuron found in the spinal cord would have distinct genotype, appearance and functional characteristics to that of a neuron found in the brain and the neuron would be surrounded by its own distinct extracellular matrix.

Further, there has been found to be a worldwide increase in the prevalence of neurodevelopmental disabilities, such as autism, attention-deficit hyperactivity disorder, and other cognitive impairments, which has brought concern regarding the role of exposure to environmental toxicants in causing induced developmental neurotoxicity (DNT). The developing brain is particularly vulnerable to the environmental toxicants such as chemical exposure, and the widespread presence of industrial chemicals in the environment creates multiple avenues for insult. Progress in the last three decades to understand the hazards of exposure to a small set of developmental neurotoxicants has been limited. Furthermore, little to no effort has been spent to characterize the potential developmental neurotoxic hazards for the litany of other chemicals in common use. It is now estimated that of the over 80,000 chemicals currently available, only 200 have undergone DNT testing according to established guidelines. Testing for DNT continually depends on the use of animal models, which cost millions of dollars per chemical and take months to years to complete. As such, the long-term use of these models for testing is not viable for evaluating the increasingly long list of environmental neurotoxins, driving a need to establish new alternatives for DNT testing.

Currently, to model the surrounding matrix, which is normally rich in laminin, fibronectin and other such large extracellular proteins—matrices such as MATRIGEL®, collagen, fibronectin and laminin are used, often as a thin coating, on stiff materials such as tissue culture plastic or glass. Specifically, neural progenitor cells, astrocytes and neurons are plated onto these extracellular matrices (ECMs) and, by subtle changes in external growth factor composition and medium, directed to promote adhesion, proliferation, differentiation and maturation. More particularly, neural progenitor cells are normally grown on MATRIGEL®, which is a poorly defined substrate, with high lot-to-lot variation and lacks ease of use. Astrocytes and neurons are normally grown on laminin coated tissue culture plastic, thereby exposing the cells to a highly stiff substrate that is not representative of the native microenvironment. Collagen and fibronectin have additionally been used in various combinations with both MATRIGEL® and laminin and themselves to demonstrate a suitable environment to the neuronal population.

Accordingly, there exists a need for biomaterial compositions and to methods for preparing the biomaterial compositions capable of supporting survival and growth of neural cells in culture, and particularly, to provide specific molecules that promote cellular expansion, cellular differentiation and regulate cellular behavior. Further, the neurite outgrowth assay has been previously demonstrated to serve as a suitable measure for induced neurotoxicity (NT) [Citation]. The formation of a neural network is a crucial step in the nervous system development during which neurons extend long cytoskeletal processes, known as neurites, to ultimately form a mature neural network [A robust and reproducible]. Interruption of this process has been shown to be present in many nervous system disorders including

### BRIEF DESCRIPTION OF THE DISCLOSURE

The present disclosure relates generally to biomaterial compositions and methods for using the biomaterial compositions. More particularly, the present disclosure relates to hydrogel compositions and methods for promoting neural cell expansion and neural cell differentiation using the hydrogel compositions.

In accordance with the present disclosure, hydrogel compositions and methods for preparing the hydrogel compositions to support survival and growth of neural cells in culture have been discovered. The hydrogel compositions of the present disclosure can also be used for two-dimensional (2D) and three-dimensional (3D) cell culture. The hydrogel compositions of the present disclosure can further be used for two-dimensional and three-dimensional enrichment of biomolecules such as, for example, biomolecules to cell surfaces using soluble factor binders. The hydrogel compositions further offer design control over both composition components and hydrogel substrate stiffness, allowing for attachment with phenotypes consistent with those offered by the specific native microenvironments of various neural subpopulations. That is, the compositions can be tailored to the specific neural subpopulation and provide the optimum conditions for cell viability and growth.

In one aspect, the present disclosure is directed to a hydrogel composition for promoting neural cellular expansion and/or differentiation. The hydrogel comprises: from about 20 mg/mL to about 100 mg/mL of a polyethylene glycol, at least about 0.125 mM cell adhesion peptide, and a soluble factor binder, and wherein the hydrogel composition has a degree of crosslinking ranging from about 50% to about 70%.

In another aspect, the present disclosure is directed to a method of promoting cellular expansion, the method comprising: preparing a hydrogel composition, wherein the hydrogel composition comprises from about 20 mg/mL to about 100 mg/mL of a polyethylene glycol, at least about 0.125 mM cell adhesion peptide, and a soluble factor binder, and wherein the hydrogel composition has a degree of crosslinking ranging from about 50% to about 70%; contacting a cell with the hydrogel composition; and culturing the cell.

In yet another aspect, the present disclosure is directed to a method for neurotoxicity screening of cells, the method comprising: preparing a hydrogel composition, wherein the hydrogel composition comprises from about 20 mg/mL to about 100 mg/mL of a polyethylene glycol, at least about 0.125 mM cell adhesion peptide, and a soluble factor binder, and wherein the hydrogel composition has a degree of crosslinking ranging from about 50% to about 70%; contacting a cell with the hydrogel composition; culturing the cell to form a network; contacting the network with a candidate neurotoxicant; and analyzing the growth of the network in the presence of the candidate neurotoxicant.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The disclosure will be better understood, and features, aspects and advantages other than those set forth above will become apparent when consideration is given to the following detailed description thereof. Such detailed description makes reference to the following drawings, wherein:

FIGS. 1A-1B are schematic illustrations of the steps for preparing a hydrogel array of the present disclosure.

FIG. 2A is a schematic illustration of the steps for patterning a metal-coated substrate used in the method for preparing a hydrogel array of the present disclosure.

FIG. 2B are end view drawings of the metal-coated substrate during the steps for patterning a metal-coated substrate shown in FIG. 2A.

FIG. 3 is an illustration of a hydrogel array with 64 individual hydrogel spots prepared using the methods of the present disclosure.

FIG. 4 is a graph illustrating the surface roughness of a hydrogel array as determined by atomic force microscopy.

FIG. 5 illustrates high magnification top-view images showing different shapes of individual hydrogel spots.

FIG. 6 is a side-on image showing individual hydrogel spots having different heights.

FIG. 7 is a schematic illustrating the steps for preparing a hydrogel array and further assembling the hydrogel array with a microwell add-on using the methods of the present disclosure.

FIG. 8A is a picture taken of a 384-well plate with a Corning glass bottom that was utilized in the large initial screen of Example 1. CDI forebrain derived GABA neurons were plated at a density of 5,000 cells/well (as recommended by the manufacturer) onto a precoated plate. Cells were allowed to grow for 24 hours and stained using rhodamine phalloidan (actin stain), BIII tubulin and dapi (nuclei).

FIG. 8B is the layout of the experiment assigning each hydrogel condition to a number value for a total of 192 conditions that were plated in duplicate in the 384-well plate of FIG. 8A. All plates were done in triplicate. The parameters varied in the formulation of the hydrogels as follows: concentration of PEG-NB (30, 40, or 50 mg/mL), concentration of adhesion peptide IKVAV (SEQ ID NO:37) (0, 0.125, 0.5, or 2 mM), concentration of adhesion peptide CRGDS (SEQ ID NO:2) (0, 0.25, 1, or 4 mM), identity of MMP-degradable peptide (Tryptophan or Alanine), and the degree of crosslinking in the hydrogel (50% or 70%).

FIGS. 9A-9H depicts the results of a Cell Profiler used to quantify differences between hydrogel conditions in the 384 well plate screen. For this purpose, an image analysis pipeline was created in the cell profiler to identify cellular responses via high content imaging analysis (FIGS. 9A-9H). Objects were identified as follows: nuclear objects labelled by dapi (FIG. 9A) were identified by background subtraction and binary image conversion (FIG. 9B) and identified as primary objects in cell profiler (FIG. 9C). This outputted the number of neural cells per condition, nuclear distance of individual neurons and % overlap of individual nuclei in the different conditions as seen in FIG. 9D, where colored objects not in blue showed overlap of neurons, resulting in difficulties in quantification and segmentation. In FIG. 9E, the neural cell actin cytoskeleton was labelled using rhodamine phalloidin and processed as follows: the image was converted to a binary image and background subtracted in (FIG. 9F) secondary objects were identified via thresholding and associated with primary nuclear objects identified in FIG. 9C to identify individual neurons and their associated neuronal processes. This allowed quantification of the following parameters: (FIG. 9A) neuronal area (FIG. 9B) cytoplasm overlap (FIG. 9C) major and minor axis length. For branch point analysis and quantification of the longest neurite, images were skeletonized and quantified using the neuron measure function in Cell Profiler (FIG. 9H).

FIGS. 10A-10E depict heat maps corrected from the results of each parameter of the high throughput software; that is, using the results from each parameter of the high throughput software, the data was reorganized into a template matching FIG. 8B and then correlated to heat maps, which stratifies the data by 15% tiers. Each color on the map correlates to a 15% tier with the highest values indicated by green and the lowest values indicated in red. The gaps in the heat maps are primarily through outliers found in the initial screen. If a value was beyond or below 200% of the average value in the map, it was deemed an outlier and not counted in the analysis. A total of 8 conditions were considered outliers in this Example and subsequently not utilized in the heat map analysis. FIG. 10A correlates to the total neuron area parameter; FIG. 10B correlates to number of dendritic processes; FIG. 10C correlates to the percent of overlap between nuclei; FIG. 10D correlates to the longest measured neuron; and FIG. 10E correlates to a condition's overall score in the ranking.

FIGS. 11A-11E: FIG. 11A depicts the top 10% of conditions resulting from the screening: (top) top 10% conditions colored in blue mapped to condition number; (bottom) top 10% conditions colored in blue mapped to score from multi-parametric scores cumulative multi (FIGS. 11B-11E). Comparison of conditions screened versus gold standard controls. (FIG. 11B) Conditions identified show less clustering of individual neurons than laminin or MATRIGEL®, allowing for easier high content imaging and assessment of individual cellular effects on neurons. (FIG. 11C). Increase of number of dendritic processes versus that of gold standard controls (FIG. 11D) Materials support longer neural outgrowth than MATRIGEL® controls in first 24 hours and larger cell somas than MATRIGEL® (FIG. 11E), indicating greater adhesion and cell spreading on hydrogel surfaces than that of MATRIGEL®.

FIG. 12A is a schematic of the hydrogel composition used in Example 2.

FIG. 12B depicts incubation of cells on the hydrogel composition and control substrate (PLL/laminate).

FIG. 12C depicts cell adhesion to the synthetic hydrogel composition

FIGS. 13A-13D depict the effect of treatment of iCell neurons to DMSO or 100 μM 5HPP on neurite branching (FIGS. 13A & 13C) and singularized cell number (FIGS. 13B & 13D).

FIGS. 14A-14C depict the comparison of compounds affecting neurite growth specifically or unspecifically on synthetic scaffold. iCell Neurons were treated with compounds following a 120-hour growth period and exposed for 120 hours with media replenished daily. All data points are mean±SEM. FIG. 14A: Dexamethasone. FIG. 14B: Colchicine. FIG. 14C: Carbamazepine. *p<0.05 versus untreated control, # p<0.05 versus viable cells at that concentration.

FIG. 15 depicts the effects to known developmental neurotoxicants of iCell Neuron networks on synthetic hydrogels as compared to networks on PLL/Laminin and MATRIGEL® substrates.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the scope of the disclosure as defined by the appended claims.

### DETAILED DESCRIPTION OF THE DISCLOSURE

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

In accordance with the present disclosure, methods for preparing biomaterial compositions for promoting cellular expansion and differentiation have been discovered. More particularly, the present disclosure relates to hydrogel compositions. In one aspect, hydrogel compositions can be prepared as a hydrogel array with individually controlled hydrogel spot modulus, hydrogel spot polymer density, hydrogel spot ligand identity and hydrogel spot ligand density and to methods for preparing the hydrogel arrays. In another aspect, the hydrogel compositions can be prepared as coatings such as for use on the surfaces of cell culture plates. In another aspect, the hydrogel compositions can be prepared as microcarriers in suspension culture. The hydrogel compositions of the present disclosure can be functionalized with biomolecules, are compatible with cell culture and are biocompatible. The hydrogel compositions of the present disclosure can be used to alter (e.g., enhance, inhibit and change) cell function, and in particular, cellular expansion, maturation and differentiation of neuronal cells.

As known by those skilled in the art, a hydrogel composition is a network of polymer chains that are hydrophilic in which a polymeric material and water are in an equilibrated form. The hydrogel composition is formed using unpolymerized starting components. The polymeric material can be, for example, a natural polymer material, a synthetic polymer material and combinations thereof.

The methods for preparing hydrogel compositions of the present disclosure advantageously allow for the direct incorporation of peptides into the hydrogel network during polymerization by including a cysteine in the amino acid sequence during synthesis, which allows for eliminating the need for post-synthetic modifications. In this way, peptides can be utilized as crosslinkers by including cysteine on each end or they can be incorporated as pendant groups, which can be pre-coupled to the polymer backbone and mixed in varying combinations or incorporated during polymerization for simplicity.

### Hydrogel Compositions and Methods for Preparing Hydrogel Compositions

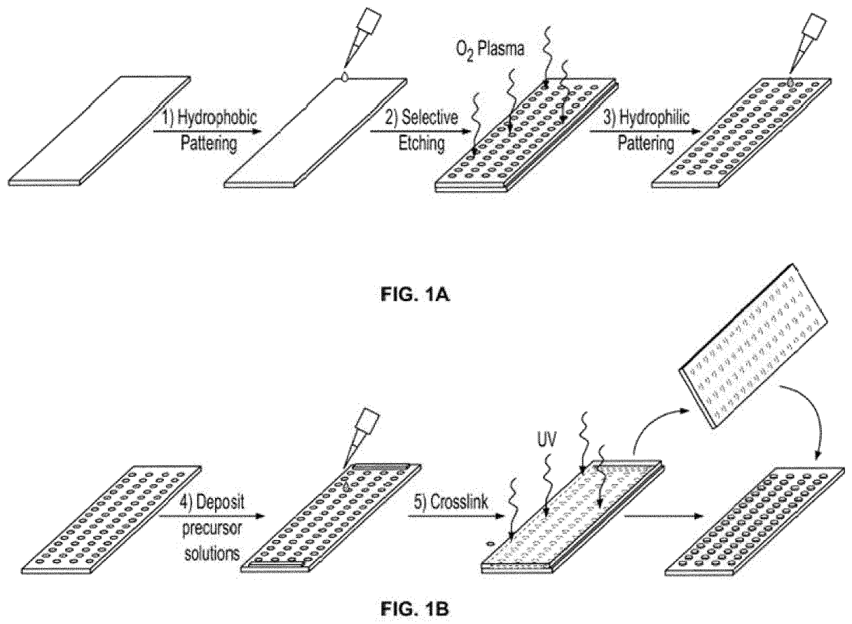

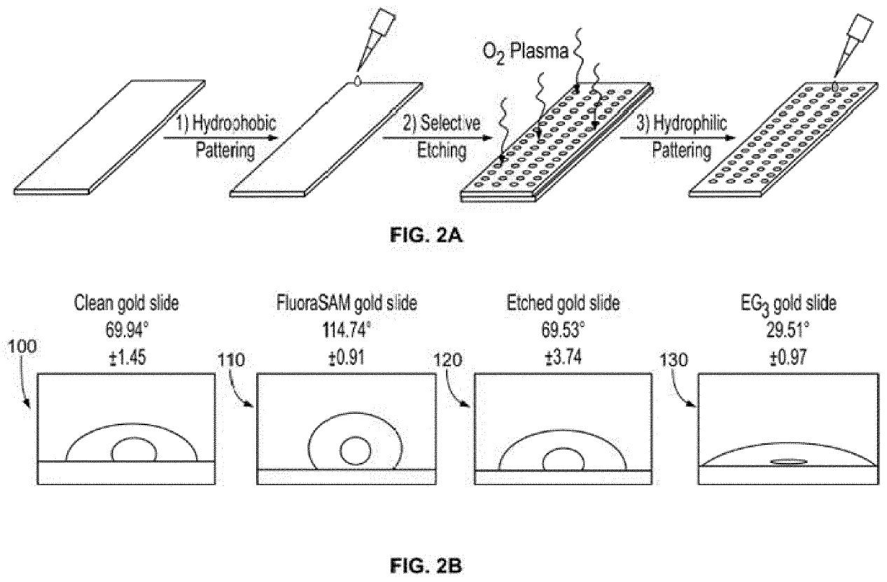

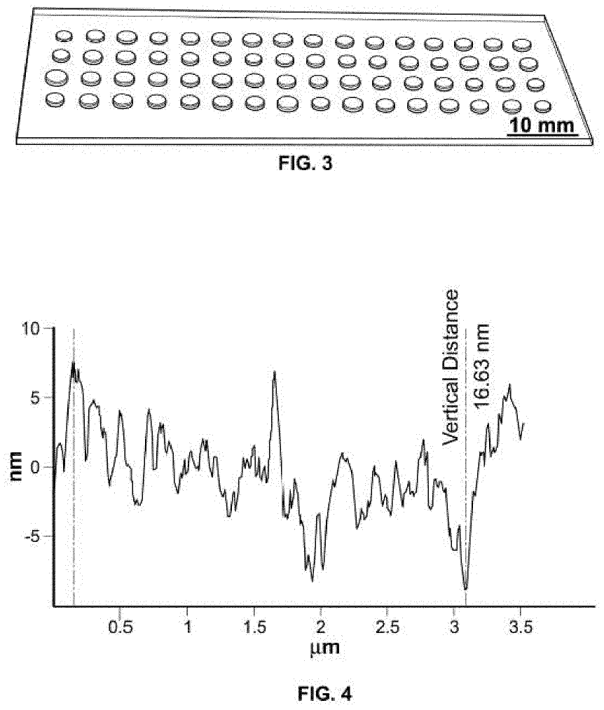

The present disclosure is generally directed to methods for preparing a hydrogel composition and use of the resulting compositions. When used to prepare a hydrogel array, the preparation methods generally include contacting a hydrogel precursor solution with a substrate, wherein the substrate includes a hydrophobic region and a hydrophilic region; placing a surface-modified substrate onto the hydrogel precursor solution such that the hydrogel precursor solution is located between the substrate and the surface-modified substrate; polymerizing the hydrogel precursor solution; and separating the surface-modified substrate from the substrate, to result in the hydrogel array. (See, FIGS. 1A-1B). Thus, the polymer hydrogel precursor solution polymerizes between the substrate and the surface-modified substrate and the resultant hydrogel transfers with the surface-modified substrate such that the surface-modified substrate includes the hydrogel array. In one embodiment, the hydrogel array can be patterned to include an array of hydrogel spots surrounded by a hydrogel-free background as described in more detail below. In another embodiment, the hydrogel array can be patterned such that an array of hydrogel-free spots (or pools) is formed within a hydrogel background as described in more detail below.

In hydrogel arrays having hydrogel spots, the resultant hydrogel array can be patterned to result in differential wettability to define the geometry of each hydrogel spot and confine the contents of each hydrogel spot of the array, as well as define the spatial pattern of each hydrogel spot in the array in relation to neighboring spots. As used herein, “spot” refers to an area, a place, or a region of the substrate that include hydrogel. “Hydrogel free spots” refers to an area, a place, or a region of the substrate that is substantially, or even completely, free of hydrogel; that is the spot does not include hydrogel. This is particularly useful for preparing hydrogel arrays for use with common microarray add-ons of different sizes and dimensions consistent with those of common multi-well plates (e.g., 96-well plates, 384-well plates, etc.) This is also useful for use with multichannel pipettes for enhanced-throughput cell culture, media exchange, and the like. The individual hydrogel spots of the array can have any desired shape (see e.g., FIG. 5). For example, the shape can be circular, round, oval, quatrefoil, rectangular, triangular, star-shaped, diamond-shaped, combinations thereof, and the like. Patterns of hydrogel spots may also be created in rows, spirals, circles, squares, rectangles, combinations thereof, and the like. The shape of the individual hydrogel spot can be varied by changing the pattern of the stencil used for etching during patterning of the patterned substrate.

In hydrogel arrays having hydrogel-free spots, the individual hydrogel-free spots can have any desired shape. For example, the shape can be circular, round, oval, quatrefoil, rectangular, triangular, star-shaped, diamond-shaped, combinations thereof, and the like. Patterns of hydrogel-free spots may also be created in rows, spirals, circles, squares, rectangles, combinations thereof, and the like. The shape of the individual hydrogel-free spot can be varied by changing the pattern of the stencil used for etching during patterning of the patterned substrate.

The upper size limit of the hydrogel array depends on the dimensions of the patterned substrate and/or the dimensions of the surface-modified substrate. The resultant hydrogel array can also be patterned to result in individual hydrogel spots and hydrogel-free spots having any desired sizes. The size and shape of the individual hydrogel spot and hydrogel-free spot can be varied by changing the pattern of the stencil used for etching during patterning of the patterned substrate. Suitable individual hydrogel spot size of the hydrogel array can be small enough to accommodate a single cell, but also large enough to accommodate many cells, for example Thus, the individual hydrogel spot size of the hydrogel array can have any desired diameter. Particularly suitable individual hydrogel spot sizes of the hydrogel array can be about 10 μm and larger in diameter.

A patterned substrate can be prepared by creating hydrophobic regions and hydrophilic regions formed by self-assembled monolayers (SAMs), such as described in U.S. patent application Ser. No. 14/339,938 (published as U.S. Publication No. 2015/0293073), filed on Jul. 24, 2014, herein incorporated by reference to the extent it is consistent herewith. Suitable substrates for forming self-assembled monolayers are known to those skilled in the art and can be, for example, metal-coated substrates, silicon substrates, diamond substrates, polydimethylsiloxane (PDMS) substrates, and the like (as described in Love et al., Chem. Rev. 2005, 105:1103-1169, for example, which is hereby incorporated by reference to the extent its disclosure is consistent with the present disclosure). The patterned substrate can be prepared, for example, by forming regions with differential wettability on a substrate by immersing the substrate in a perfluorinated alkanethiol solution to allow perfluorinated alkanethiolate self-assembled monolayers (fluoraSAMs) to form. To form hydrophilic regions, a stencil can be placed on the fluoraSAMs metal-coated substrate to selectively protect regions of the fluoraSAMs metal-coated substrate from plasma etching. Exposed regions of the fluoraSAMs substrate can then be etched by oxygen plasma treatment to form etched fluoraSAMs in the substrate. The substrate is then immersed in a hydroxyl-terminated alkanethiol solution to form a hydrophilic alkanethiolate SAM (EG₃SAM) in the etched regions of the substrate. The resulting patterned substrate possesses differential wettability based on the hydrophobic SAMs and hydrophilic SAMs.

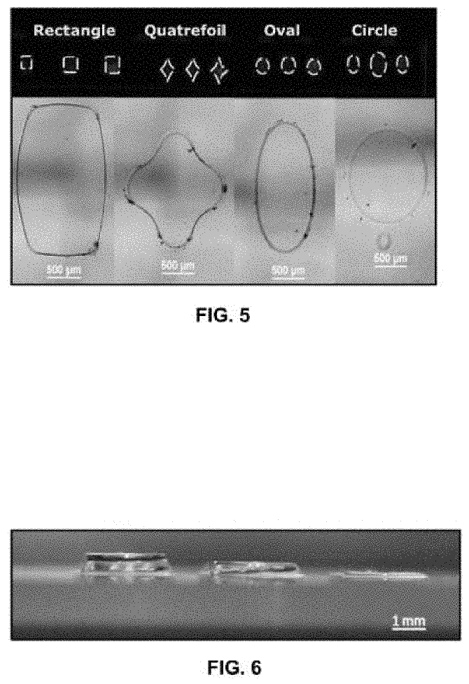

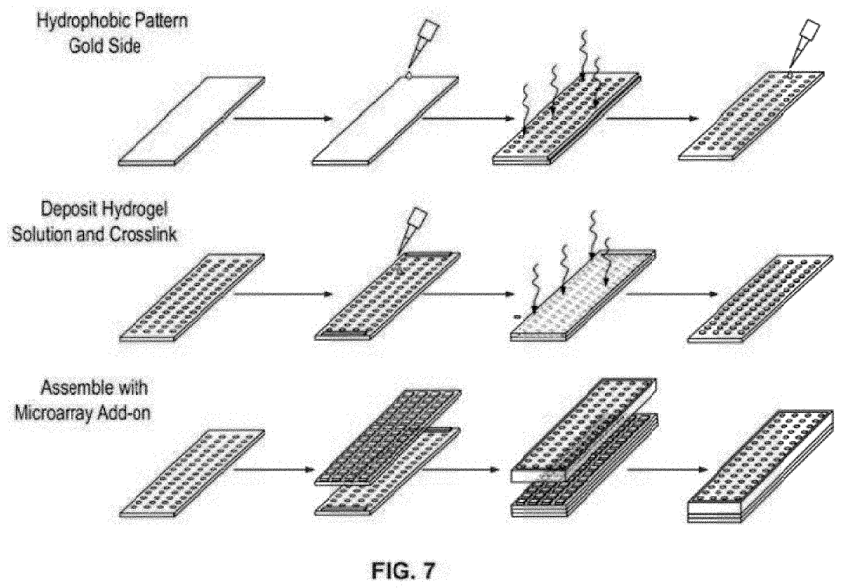

The method can further include placing a spacer between the patterned substrate and the surface-modified substrate. The spacer placed onto the patterned substrate while performing the method functions to define the height (or thickness) of the hydrogel forming the hydrogel array. A spacer may be particularly desirable when preparing higher (i.e., thicker) hydrogel arrays. Thus, the hydrogel array can have any desirable height (see e.g., FIG. 6). Suitable heights of the hydrogel array can be from about 20 micrometers (μm) to about 1 millimeter, however, hydrogel arrays can be made much higher than 1 millimeter if desired. The spacer also functions to prevent direct contact between the surface of the patterned substrate and the surface-modified substrate during formation of the hydrogel. The spacer used in the method can be any suitable material known to those skilled in the art. A particularly suitable spacer can be, for example, polydimethylsiloxane (PDMS). The height the hydrogel array can be determined, for example, using a microscope to focus from the top of the hydrogel down to the substrate, using a microscope to focus from the substrate up to the top of the hydrogel, and by measuring the surface roughness of a hydrogel array as determined by atomic force microscopy (see e.g., FIG. 4).

The preparation method further includes contacting a hydrogel precursor solution with the patterned substrate. In particular, the hydrogel precursor solution is contacted with the hydrophilic regions of the patterned substrate. The hydrophobic regions of the patterned substrate serve as a barrier between neighboring hydrophilic regions and also allow for the isolation of each hydrophilic region. The hydrogel precursor solution can be, for example, a combination of a polymer and a multifunctional polymer crosslinker.

When used as a hydrogel coating composition, preparation methods generally include contacting the hydrogel precursor solution as described above with a substrate to be coated (e.g., surface of a cell culture plate).

Suitable polymers for use in the hydrogel precursor solution are known by those skilled in the art and can include, for example, poly(ethylene glycol), hyaluronic acid, gelatin, collagen, MATRIGEL®, dithiol polymers (e.g., acrylamide), click-based composite hydrogels (as discussed in Polizzotti et al. Biomacromolecules 2008, 9:1084-1087, which is hereby incorporated by reference to the extent its disclosure is consistent with the present disclosure), poly(ethylene glycol)-diacrylate, poly(ethylene glycol)-vinyl sulfone, and the like. Particularly suitable polymers can be, for example, poly(ethylene glycol). Particularly suitable polymers can be, for example, functionalized polymers. Functionalization of the polymer can be confirmed with ¹H nuclear magnetic resonance spectroscopy, mass spectroscopy, Elman's reagent, UV-Vis spectroscopy, infrared spectroscopy, and other methods known to those skilled in the art, for example.

A particularly suitable functionalized polymer can be, for example, eight-arm poly(ethylene glycol) with terminal hydroxyl (—OH) groups (commercially available from JenKem Technology USA, Allen, Tex.) that is functionalized with norbornene. Eight-arm poly(ethylene glycol) can be functionalized with norbornene as described in Fairbanks et al. (Adv. Mater. 2009, 21:5005-5010).

Other particularly suitable polymers are poly(ethylene glycols) that may be functionalized using click chemistry. “Click” chemistry is an extremely versatile method for chemically attaching biomolecules, which is used to describe the [3+2] cycloaddition between alkyne and azide functional groups. Azides and alkynes are largely inert towards biological molecules and aqueous environments, which allows the use of the Huisgen 1,3-dipolar cycloaddition to yield stable triazoles that are very difficult to oxidize or reduce. Both the copper(I)-catalyzed and copper-free strained-alkyne variant reactions are mild and very efficient. These reactions can also be performed in small volumes of aqueous solutions, are insensitive to oxygen and water, and robust to functional groups on peptides. Click chemistry allows for selectivity in conjugation reactions in biological samples such as, for example, oligonucleotides and proteins. Particularly suitable reagents for click chemistry are commercially available from Laysan Bio Inc. (Arab, Ala.).

Generally, the hydrogel precursor solutions include concentrations of polymer of up to, and including, 200 mg/mL, including from about 36 mg/mL to about 160 mg/mL, and including from about 36 mg/mL to about 70 mg/mL.

Suitable multifunctional polymer crosslinkers for use in the hydrogel precursor solution are known by those skilled in the art. In particular, the multifunctional crosslinker can be, for example, a bifunctional polymer crosslinker and a multifunctional polymer crosslinker (n>=2) and terminated with a functional group that can form a covalent bond with the polymer of the hydrogel precursor solution. Particularly suitable bi-functional polymer crosslinkers and multifunctional polymer crosslinkers can be, for example, polyethylene glycol dithiol (PEG-DT), protease-degradable crosslinkers and multi-arm poly(ethylene glycol) terminated with thiol (e.g., 4-arm PEG terminated with thiol). Suitable protease-degradable crosslinkers can be, for example, matrix metalloproteinase (MMP)-degradable crosslinkers as described in Nagase and Fields (Biopolymers 1996, 40:399-416, which is hereby incorporated by reference to the extent it is consistent with the present disclosure). More particularly, suitable MMP-degradable crosslinking peptides for use in the hydrogel precursor solution include KCGGPQGIWGQGCK (SEQ ID NO:27) and KCGGPQGIAGQGCK (SEQ ID NO:28).

The hydrogel precursor solution can further include an initiator. As known by those skilled in the art, hydrogel polymerization can occur in the absence of an initiator. An initiator can, however, induce polymerization and/or decrease the polymerization rate. Suitable initiators are known to those skilled in the art and can be, for example, chemical initiators and photoinitiators. Particularly suitable photoinitiators can be, for example, IRGACURE 2959 photoinitiator (commercially available from Ciba/BASF, Ludwigshafen, Germany) and Eosin Y. Polymerization to form the hydrogel can also be performed by temperature change.

In another aspect, the hydrogel precursor solution can include a cell adhesion peptide. As used herein, a “cell adhesion peptide” refers to an amino acid sequence obtained from an adhesion protein to which cells bind via a receptor-ligand interaction. Varying the cell adhesion peptide and concentrations thereof in the solution allow for the ability to control the stability of the cellular attachment to the resulting hydrogel composition. Suitable cell adhesion peptides include, for example, RGD, RGDS (SEQ ID NO:1), CRGDS (SEQ ID NO:2), CRGDSP (SEQ ID NO:3), PHSRN (SEQ ID NO:4), GWGGRGDSP (SEQ ID NO:5), SIDQVEPYSSTAQ (SEQ ID NO:6), GRNIAEIIKDI (SEQ ID NO:7), DITYVRLKF (SEQ ID NO:8), DITVTLNRL (SEQ ID NO:9), GRYVVLPR (SEQ ID NO:10), GNRWHSIYITRFG (SEQ ID NO:11), GASIKVAVSADR (SEQ ID NO:12), GTTVKYIFR (SEQ ID NO:13), GSIKIRGTYS (SEQ ID NO:14), GSINNNR (SEQ ID NO:15), SDPGYIGSR (SEQ ID NO:16), YIGSR (SEQ ID NO:17), GTPGPQGIAGQGVV (SEQ ID NO:18), GTPGPQGIAGQRVV (SEQ ID NO:19), MNYYSNS (SEQ ID NO:20), KKQRFRHRNRKG (SEQ ID NO:21), CRGDGGGGGGGGGGGGGPHSRN (SEQ ID NO:29), CPHSRNSGSGSGSGSGRGD (SEQ ID NO:30), Acetylated-GCYGRGDSPG (SEQ ID NO:31), CRDGS (SEQ ID NO:32), cyclic RGD {Fd}C (SEQ ID NO:33), RKRLQVQLSIRT (SEQ ID NO:36), IKVAV (SEQ ID NO:37), YIGSR (SEQ ID NO:38), KRTGQYKL (SEQ ID NO:39), TYRSRKY (SEQ ID NO:40), KRTGQYKLGSKTGPGQK (SEQ ID NO:41), QAKHKQRKRLKSSC (SEQ ID NO:42), SPKHHSQRARKKKNKNC (SEQ ID NO:43), XBBXBX, wherein B=basic residue and X=hydropathic residue (SEQ ID NO:44), XBBBXXBX, wherein B=basic residue and X=hydropathic residue (SEQ ID NO:45), and RGDSP (SEQ ID NO:46).

The concentration of cell adhesion peptide in the hydrogel precursor solution will depend on the specific cell adhesion peptide being used as well as the other components in the hydrogel precursor solution. Typically, however, the hydrogel precursor solution includes from about 0.125 mM to about 4 mM cell adhesion peptide, including from about 0.25 mM to about 2 mM cell adhesion peptide. In one suitable embodiment, the cell adhesion peptide is CRGDS (SEQ ID NO:2), and the hydrogel precursor solution includes from about 0.25 mM to about 4 mM CRGDS (SEQ ID NO:2). In another suitable embodiment, the cell adhesion peptide is a cyclic RGD, and the hydrogel precursor solution includes from about 0.125 mM to about 2 mM cyclic RGD, particularly cyclic RGD{Fd}C (SEQ ID NO:33). In yet another suitable embodiment, the cell adhesion peptide is IKVAV (SEQ ID NO:37), and the hydrogel precursor solution includes from about 0.125 mM to about 2 mM IKVAV (SEQ ID NO:37).

In another aspect, the hydrogel precursor solution can include a soluble factor binder. In one aspect, a peptide for binding a soluble factor contained in a cell culture medium is included in the hydrogel precursor solution. The density (concentration) of the soluble factor binder in a hydrogel composition can be controlled by altering the concentration of the soluble factor binder in the hydrogel precursor solution. Examples of particularly suitable soluble factor binders are provided in Table 1, below.

TABLE 1
Soluble factor binder peptide sequences for
hydrogel compositions.

| | | SEQ |
|---|---|---|
| | | ID |
| Name/Source | Sequence | NO: |
| Vascular | GGGKLTWQELYQLKYKGI | 22 |
| Endothelial | | |
| Growth Factor- | | |
| Receptor | | |
| Binding | | |
| Peptide | | |
| Vascular | KLTWQELYQLKYKGI | 23 |
| endothelial | | |
| growth factor | | |
| receptor | | |
| binding | | |
| peptide (VR-BP) | | |
| Bone | KIPKASSVPTEL | 24 |
| morphogenetic | | |
| protein-2 | | |
| (BMP-2) | | |
| receptor | | |
| binding | | |
| peptide | | |
| Bone | KIPKASSVPTELSAISTLYL | 25 |
| morphogenic | | |
| protein | | |
| receptor- | | |
| binding peptide | | |
| Heparin | KRTGQYKL | 26 |
| proteoglycan- | | |
| binding peptide | | |
| (HPG-BP) | | |
| VEGF binding | CE{Fd}{Ad}{Yd}{Ld}IDFNWEYPASK | 34 |
| peptide | | |
| Scrambled VEGF | CD{Ad}PYN{Fd}EFAWE{Yd}VIS{Ld}K | 35 |
| binding peptide | | |

The concentration of soluble factor binder in the hydrogel precursor solution will depend on the specific soluble factor binder being used as well as the other components in the hydrogel precursor solution.

In another aspect, the hydrogel precursor solution can further include a cell. Suitable cells are known to those skilled in the art and can include any neuronal cell known in the art, for example, an embryonic stem cell-derived neuron, an embryonic stem cell-derived neural progenitor cell, an embryonic stem cell-derived astrocyte, an embryonic stem cell-derived microglial cell, an induced pluripotent stem cell-derived neural progenitor cell, an induced pluripotent stem cell-derived astrocyte, an induced pluripotent stem cell-derived microglial cell, a neuron, and combinations thereof.

In another aspect, the hydrogel precursor solution can further include a microsphere carrier (i.e., microcarrier). Microsphere carriers can contain molecules such as, for example, cells, biomolecules, dyes and other molecules known to those skilled in the art. Microspheres can be degradable microspheres that dissolve or degrade to release the contents of the microsphere.

Once prepared, the hydrogel precursor solution is contacted with a substrate (e.g., a patterned surface-modified substrate, surface of a cell culture plate, etc.).

When used on a patterned surface-modified substrate, the surface-modified substrate can be, for example, mica, glass, silicon, diamond and metal oxide surfaces. The surface-modified substrate can be prepared, for example, by functionalizing a surface such as a glass coverslip having a silane monolayer. A particularly suitable surface-modified substrate can be, for example, a glass slide. A particularly suitable method for functionalizing the substrate can be, for example, silanization. The substrate can be surface-modified by activating both sides of the surface in oxygen plasma treatment. Oxygen plasma treatment can increase the number of activated hydroxyl groups on the surface of the substrate. As known by those skilled in the art, a silane monolayer can be prepared with an alkoxysilane that is dissolved in an anhydrous organic solvent such as, for example, toluene. Other suitable alkoxysilanes can be for example, aminosilanes, glycidoxysilanes and mercaptosilanes. Particularly suitable aminosilanes can be, for example, (3-aminopropyl)-triethoxysilane, (3-aminopropyl)-diethoxy-methylsilane, (3-aminopropyl)-dimethyl-ethoxysilane and (3-aminopropyl)-trimethoxysilane. Particularly suitable glycidoxysilanes can be, for example, (3-glycidoxypropyl)-dimethyl-ethoxysilane. Particularly suitable mercaptosilanes can be, for example, (3-mercaptopropyl)-trimethoxysilane and (3-mercaptopropyl)-methyl-dimethoxysilane. Other suitable silanes are commercially available (Sigma Aldrich, St. Louis, Mo.). Preparation of a surface-modified silane substrate can be performed using any silane having a terminal functional group that can participate in click chemistry as described herein. For example, mercaptosilane contains a terminal thiol that can react with the norbornene of the PEG-norbornene. Other suitable functional surface-modified silane substrates can be, for example, acrylates and methacrylates. Following surface-modification of the substrate, non-adhesive self-assembled monolayers are formed on the surface-modified substrate.

After contacting the substrate with the hydrogel precursor solution, the method includes polymerizing the hydrogel precursor solution such that polymerized hydrogel attaches (i.e., is coupled) to the substrate.

In one embodiment, the method can be used to form an array having “spots” or “islands” of hydrogel (referred to herein as “hydrogel spots”) that are surrounded by a background that is substantially free, and even completely free, of hydrogel (“hydrogel-free”). In this embodiment, the hydrogel-free background corresponds to the hydrophobic regions of the patterned substrate and the hydrogel spots correspond to the hydrophilic regions of the patterned substrate. Referring to FIG. 1, the circles would represent the hydrogel spots that would be surrounded by a hydrogel-free region in this embodiment.

In another embodiment, the method can be used to form an array having hydrogel-free pools surrounded by a background of hydrogel (referred to herein as “a hydrogel background”). Referring to FIGS. 1A & 1B, the circles would represent the hydrogel-free pools that would be surrounded by the hydrogel background in this embodiment.

In another aspect, the present disclosure is directed to hydrogel compositions including hydrogel spots having variable moduli, variable shear moduli, variable ligand identities, variable ligand densities and combinations thereof. Hydrogel compositions having variable moduli, variable shear moduli, variable ligand identities, variable ligand densities and combinations thereof can be prepared according to the methods described herein above.

Suitable ligands are known to those skilled in the art and can be, for example, any biomolecule containing a cysteine and/or functionalized with a thiol. Thiol-functionalizing of ligands can be performed using commercially available kits (e.g., Traut's Reagent (2-iminothiolane.HCl), Thermo Fischer Scientific, Rockford, Ill.). Suitable ligands can be, for example, proteins, peptides, nucleic acids, polysaccharides, lipids, biomimetic materials and other molecules, and combinations thereof. Particularly suitable proteins can be, for example, adhesion proteins. Particularly suitable adhesion proteins can be, for example, fibronectin, cadherin and combinations thereof. Particularly suitable peptides can be, for example, cell adhesion peptides and/or soluble factor binders, as described herein above.

Suitably, the hydrogel compositions of the present disclosure include combinations of cell adhesion peptides and soluble factor binders that are suspected of binding or interacting with a cell to affect cell attachment, spreading, migration, maturation, proliferation, differentiation, and formation of cellular structures (e.g., tubules).

Hydrogel compositions may further include variable moduli. Hydrogel compositions can have a range of stiffness (expressed herein as substrate elastic moduli). For example, hydrogels with different moduli can be prepared by changing the concentration of the polymer and/or changing the stoichiometric ratio of the multifunctional polymer (e.g., the bifunctional polymer thiol-polyethylene glycol-thiol (SH-PEG-SH)) to polymer ratio in the hydrogel precursor solution. Suitable ratios can be from about 1:1 to about 4:1 (molar ratio).

In another aspect, the patterned hydrogel array can be further assembled with a microarray add-on whereby the patterned hydrogel array is prepared with dimensions to accommodate add-ons of any size. Suitable microarray add-ons are commercially available (Grace Bio Labs, Bend, Oreg.). A microarray add-on can allow for the isolation of each individual hydrogel spot and hydrogel-free pool of the hydrogel array such that soluble factor presentation can be controlled. The microarray add-on can include the same number of openings as the number of individual hydrogel spots and hydrogel-free pools of the hydrogel array such that each hydrogel spot and hydrogel-free pool can be independently interrogated with soluble factor presentation. Alternatively, the microarray add-on can have larger openings that can accommodate more than one individual hydrogel spot and more than one individual hydrogel-free pool. For example, a microarray add-on can have openings large enough to accommodate a single hydrogel spot or a single hydrogel-free pool.

### Methods of Using the Hydrogel Compositions

In another aspect, the present disclosure is directed to methods of using the hydrogel compositions to promote cellular expansion, maturation and cellular differentiation. Generally, the methods include preparing the hydrogel compositions; contacting a cell with the hydrogel compositions; and culturing the cells. The hydrogel compositions are prepared as described above and typically include a polymer (e.g., a polyethylene glycol functionalized with norbornene), a multifunctional polymer crosslinker (e.g., MMP-degradable crosslinking peptide, non-degradable PEG-dithiol crosslinker), and a cell adhesion peptide as described more fully above.

The method further includes contacting a cell with the hydrogel composition. As used herein, “contacting a cell” refers to seeding the cells with the purpose of culturing the cells. As known by those skilled in the art, a cell suspension is typically transferred to a substrate and cells are given sufficient time to adhere to the substrate.

In another embodiment, cells can be incorporated into the hydrogel using a hydrogel precursor solution that includes the polymer, the crosslinker, the cell adhesion peptide, and the cell.

In yet another embodiment, cells can be adhered to the hydrogel once the hydrogel is prepared.

The cells are then cultured for a desired time such as, for example, about one hour to about 30 days. After the desired time, cells can be analyzed by microscopy such as, for example, immunofluorescence microscopy, phase contrast microscopy, light microscopy, electron microscopy and combinations thereof. Cells can be analyzed for cell attachment, cell spreading, cell morphology, cell proliferation, cell migration, cell expansion, cell differentiation, protein expression, cell-to-cell contact formation, sprouting, tubulogenesis, formation of structures, and combinations thereof.

Suitable cells can be any cell known by those skilled in the art. Particularly suitable cells can include, for example, an embryonic stem cell, an embryonic stem cell-derived neuron, an embryonic stem cell-derived neural progenitor cell, an embryonic stem cell-derived astrocyte, an embryonic stem cell-derived microglial cell, an embryonic stem cell-derived endothelial cell, an embryonic stem cell-derived retinal pigment epithelial cell, an induced pluripotent stem cell, an induced pluripotent stem cell-derived neural progenitor cell, an induced pluripotent stem cell-derived astrocyte, an induced pluripotent stem cell-derived microglial cell, an induced pluripotent stem cell-derived endothelial cell, an induced pluripotent stem cell-derived retinal pigment epithelial cell, a mesenchymal stem cell, an umbilical vein endothelial cell, an NIH 3T3 fibroblast, a dermal fibroblast, a fibrosarcoma cell, a valvular interstitial cell, a cardiomyocyte, an induced pluripotent stem cell-derived cardiomyocyte, an endothelial progenitor cell, a circulating angiogenic cell, a neuron, a pericyte, a cancer cell, a hepatocyte, a pancreatic beta cell, a pancreatic islet cell and combinations thereof.

In one particular aspect, the cell is a neuron, for example, forebrain derived GABA neurons. In one particular aspect, when used with neurons, the hydrogel compositions include 8-arm, 20 kDa poly(ethylene glycol) (PEG) functionalized with norbornene, a MMP degradable crosslinking peptide, and a cell adhesion peptide. Particularly suitable cell adhesion peptides include immobilized RGD-containing peptides, including RGDS (SEQ ID NO:1), CRGDS (SEQ ID NO:2), Acetylated-GCYGRGDSPG (SEQ ID NO:31); cyclic {RGD(Fd)C} (SEQ ID NO:33); CRGD-(G)13-PHSRN (SEQ ID NO:29); CPHSRN-(SG)5-RGD (SEQ ID NO:30); and IKVAV (SEQ ID NO:37). Suitably, the hydrogel compositions include at least about 0.125 mM cell adhesion peptide, including from about 1 mM to about 4 mM cell adhesion peptide. Further, the hydrogel compositions may include from about 20 mg/mL to about 100 mg/mL PEG concentration, including from about 30 mg/mL to about 50 mg/mL.

In some aspects, the hydrogel compositions are prepared to include crosslinking to an extent of at least 35%, including at least 45%, and including from about 35% to about 75%, and including from about 50% to about 70%. In particularly suitable embodiments, the hydrogel compositions are prepared using degradable crosslinking peptides including alanine and/or tryptophan.

In one particularly suitable embodiment, the hydrogel composition includes 8-arm, 20 kDa poly(ethylene glycol) (PEG) functionalized with norbornene, an alanine-containing MMP degradable crosslinking peptide, and from about 0.25 mM to about 4 mM RGDS (SEQ ID NO:1), including about 0.25 mM to about 2.0 mM RGDS (SEQ ID NO:1), and including about 1.0 mM RGDS (SEQ ID NO:1). In one embodiment, the hydrogel composition is cross-linked to a degree ranging from about 50% to about 70%.

In one particularly suitable embodiment, the hydrogel composition includes 8-arm, 20 kDa poly(ethylene glycol) (PEG) functionalized with norbornene, an alanine-containing MMP degradable crosslinking peptide, and from about 0.5 mM to about 2.0 mM IKVAV (SEQ ID NO:37). In one embodiment, the hydrogel composition is cross-linked to a degree ranging from about 50% to about 70%.

In one particularly suitable embodiment, the hydrogel composition includes 8-arm, 20 kDa poly(ethylene glycol) (PEG) functionalized with norbornene, an tryptophan-containing MMP degradable crosslinking peptide, and from about 0.25 mM to about 4 mM RGDS (SEQ ID NO:1), including from about 0.25 mM to about 2.0 mM RGDS (SEQ ID NO:1), and including about 1.0 mM RGDS (SEQ ID NO:1). In one embodiment, the hydrogel composition is cross-linked to a degree ranging from about 50% to about 70%.

In one particularly suitable embodiment, the hydrogel composition includes 8-arm, 20 kDa poly(ethylene glycol) (PEG) functionalized with norbornene, an alanine-containing MMP degradable crosslinking peptide, and from about 0.125 mM to about 2.0 mM IKVAV (SEQ ID NO:37). In one embodiment, the hydrogel composition is cross-linked to a degree ranging from about 50% to about 70%.

In one particularly suitable embodiment, the hydrogel composition includes 8-arm, 20 kDa poly(ethylene glycol) (PEG) functionalized with norbornene, an tryptophan-containing MMP degradable crosslinking peptide, and from about 0.25 mM to about 4 mM RGDS (SEQ ID NO:1) and from about 0.125 mM to about 2.0 mM IKVAV (SEQ ID NO:37), including about 0.25 mM to about 2.0 mM RGDS (SEQ ID NO:1) and from about 0.5 mM to about 1.0 mM IKVAV (SEQ ID NO:37), and including about 1.0 mM RGDS (SEQ ID NO:1) and from about 0.5 mM to about 1.0 mM IKVAV (SEQ ID NO:37). In one embodiment, the hydrogel composition is cross-linked to a degree ranging from about 50% to about 70%.

Suitably, the hydrogel compositions for use with neuronal cell types include an elastic modulus in the range of from about 100 Pa to about 2 kPa for neuronal cultures.

Suitably, the hydrogel compositions for use with neurons result in increased neuronal proliferation and spreading and reduced cell aggregation and increased neuronal length over a 24-hour period along with mechanical stiffnesses matching the native neuronal tissue (e.g., brain elastic modulus is 500 Pa).

In a further aspect, the hydrogel compositions further include immobilized low molecular weight heparin. Suitably, when present, the hydrogel composition includes low molecular weight heparin in amounts ranging from about 0.1 mM to about 2 mM.

The method may further include contacting the cell with a soluble molecule by including the soluble molecule in the culture medium in which the cells are cultured. Particularly suitable soluble molecules can be growth factors and proteoglycans. Suitable growth factors can be, for example, proteins from the transforming growth factor beta superfamily, fibroblast growth factor family of growth factors, platelet derived growth factor family of growth factors and combinations thereof. Particularly suitable growth factors can be, for example, vascular endothelial growth factor, bone morphogenetic proteins, fibroblast growth factor, insulin-like growth factor and combinations thereof. Suitable proteoglycans can be, for example, proteoglycans with heparin, heparin sulfate, and/or chondroitin glycosaminoglycan side chains.

In yet other embodiments, the hydrogel compositions prepared herein can be used in methods of toxicity screening. The methods can include analyzing neural cells for sensitivity to developmental neurotoxicants. Particularly, neurite outgrowth assays have been demonstrated to serve as a suitable measure for induced neurotoxicity (NT). The formation of a neural network is a crucial step in the nervous system development during which neurons extend long cytoskeletal processes, known as neurites, to ultimately form a mature neural network Interruption of this process has been shown to be present in many nervous system disorders including autism, attention-deficit hyperactivity disorder, and other cognitive impairments.

The disclosure will be more fully understood upon consideration of the following non-limiting Examples.

### Example 1

### Materials and Methods

### PEG-Norbornene Synthesis

Eight-arm poly(ethylene glycol) (PEG) with terminal hydroxyl groups (—OH) and a molecular weight of 20 kDa was purchased from JenKem Technology USA (Allen, Tex.). Anhydrous pyridine, 4-dimethylamino)pyridine (DMAP), 5-norbornene-2-carboxylic acid, diethyl ether, and deuterated chloroform (CDCl₃, 99.8%) with 0.03% v/v tetramethylsilane (TMS) were purchased from Sigma Aldrich (St. Louis, Mo.). N,N′-Dicyclohexylcarbodiimide (DCC) and anhydrous dichloromethane (DCM) were purchased from ACROS Organics (Geel, Belgium). SNAKESKIN dialysis tubing having a 3.5K molecular weight cut-off was purchased from Thermo Fisher Scientific (Waltham, Mass.).

Eight-arm PEG-OH was functionalized with norbornene to utilize the thiol-ene chemistry for photopolymerization and immobilization of bioactive ligands (as described in Fairbanks et al. Adv. Mater. 2009, 21:5005-5010; Impellitteri et al. Biomaterials 2012, 33:3475-84; Belair and Murphy Acta Biomater. 2013; and Gould et al. Acta Biomater 2012, 8:3201-3209). The PEG-norbornene (PEG-NB) product of the functionalization reaction was filtered through a medium fritted Buchner funnel to remove salts formed during the reaction. The filtrate was then precipitated in 900 mL cold diethyl ether and 100 mL hexane. The solids were collected on qualitative grade filter paper and air dried overnight. The PEG-NB product was purified by dialysis against 4 L of dH₂O at 4° C. for 72 hours (with water change every 8 hours) using rehydrated SNAKESKIN dialysis tubing to remove residual norbornene acid and subsequently freeze dried.

Norbornene functionalization of >90% was confirmed with 1H nuclear magnetic resonance spectroscopy. Samples were prepared at 6 mg/mL in CDCl₃ with TMS internal standard. Free induction decay (FID) spectra were obtained using spectroscopy services provided by the National Magnetic Resonance Facility at Madison on a Bruker Instruments Avance III 500i spectrometer at 400 MHz and 27° C.

### Hydrogel Array Formation

Hydrogel arrays used for these experiments were composed of hydrogel spots immobilized on a 364-well plate with a Corning glass bottom that had been surface treated with 0.1 mg/ml poly-1-lysine (PLL).

Hydrogel solution was pipetted into each well (6 μl per well) to provide a uniform hydrogel surface for seeding of iPSC derived neurons. Hydrogel formulations were exposed to U.V. for four minutes, plates were rotated 180° and exposed to U.V. for a further four minutes.

### Hydrogel Spot Polymerization and Immobilization

PEG-NB was functionalized as described above. Bi-functional PEG dithiol (PEG-DT) crosslinker (3.4 kDa) was purchased from Laysan Bio (Arab, Ala.). IRGACURE 2959 photoinitiator was purchased from Ciba/BASF (Ludwigshafen, Germany) Cysteine-terminated peptides were purchased from GenScript USA (Piscataway, N.J.). Omnicure Series 1000 UV spot cure lamp (365 nm wavelength), light guide, and collimating adapter were purchased from Lumen Dynamics Group (Ontario, Canada). PDMS spacers with thickness dimensions corresponding to the desired hydrogel spot heights were fabricated using the same procedure as stated above.

Hydrogel precursor solutions were prepared by combining PEG-NB, PEG-DT, peptides, and photoinitiator and diluted to desired concentrations with phosphate buffered saline (PBS) immediately prior to hydrogel formation.

The bioactivity of each hydrogel spot (i.e., each well) was defined by both the identity and concentration of the peptides incorporated therein. Peptides used in these Examples were RGDS (SEQ ID NO:1) and IKVAV (SEQ ID NO:37). To modulate the bioactivity of each hydrogel spot, different peptides were added to the hydrogel precursor solutions and, following UV-initiated crosslinking, the resulting polymerized hydrogel networks each presented different immobilized peptides. For all well spots, a total of 0.125 mM-4.125 mM of peptides were incorporated into the hydrogel network. To concurrently change the bioactivity of the hydrogel spots via control of peptide identity and concentration, the desired concentration of the chosen bioactive peptide (containing the “RGD” sequence) was determined.

The modulus of each hydrogel spot was defined by the total concentration of PEG in the crosslinked hydrogel network. Increasingly, the concentration of PEG-NB in the hydrogel precursor solution resulted in a larger amount of PEG crosslinked into the polymerized network, which resulted in an increase in the compressive modulus.

In this Example, a hydrogel array was used to determine the effects of substrate properties on initial neuronal stem cell adhesion.

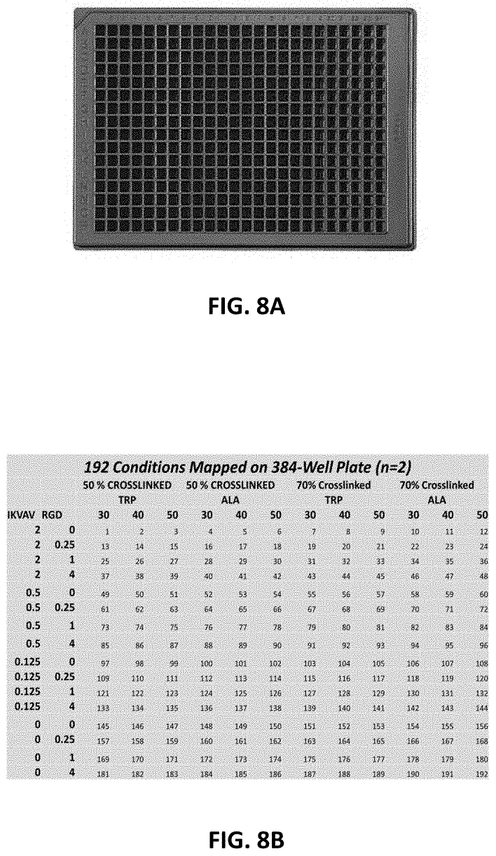

CDI forebrain derived GABA neurons were plated at a density of 5,000 cells/well onto a precoated 384-well plate as described above. Cells were allowed to grow for 24 hours and stained using rhodamine phalloidan (actin stain), BIII tubulin and dapi (nuclei). As shown in FIGS. 8A & 8B, parameters were varied in the hydrogel solutions used with the 384-well plate as follows: concentration of PEG-NB (30, 40, or 50 mg/mL), concentration of adhesion peptide IKVAV (SEQ ID NO:37) (0, 0.125, 0.5, or 2 mM), concentration of adhesion peptide CRGDS (SEQ ID NO:2) (0, 0.25, 1, or 4 mM), identity of MMP-degradable peptide (including Tryptophan or Alanine), and/or the degree of crosslinking in the hydrogel (50% or 70%). The plates were then monitored for changes in initial cell adhesion and spreading.

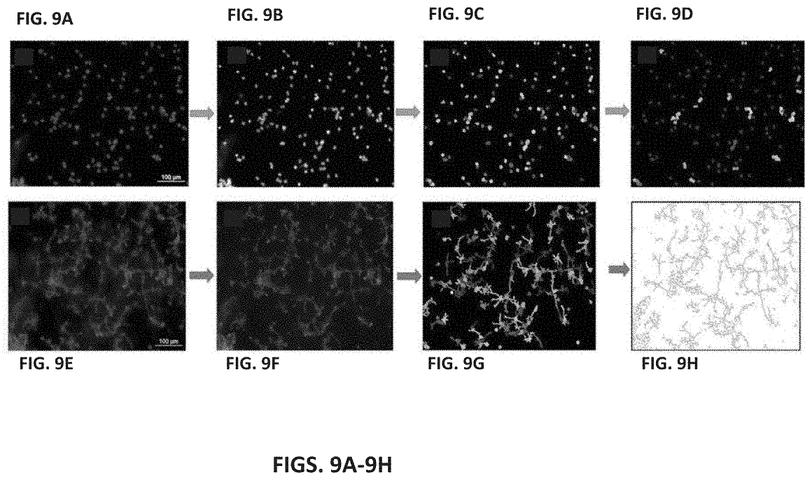

A cell profiler was then used to quantify differences between hydrogel conditions in the 384-well plate screen. For this purpose, an image analysis pipeline was created in the cell profiler to identify cellular responses via high content imaging analysis (FIGS. 9A-9H). Objects were identified as follows: nuclear objects labelled by dapi (FIG. 9A) were identified by background subtraction and binary image conversion (FIG. 9B) and identified as primary objects in cell profiler (FIG. 9C). This outputted the number of neural cells per condition, nuclear distance of individual neurons, length of neuronal processes, neuronal area, and % overlap of individual nuclei in the different conditions as seen in FIG. 9D, where colored objects not in blue showed overlap of neurons resulting in difficulties in quantification and segmentation. In FIG. 9E, the neural cell actin cytoskeleton was labelled using rhodamine phalloidin and processed as follows: the image was converted to a binary image and background subtracted in FIG. 9F, secondary objects were identified via thresholding and associated with primary nuclear objects identified in FIG. 9C to identify individual neurons and their associated neuronal processes.

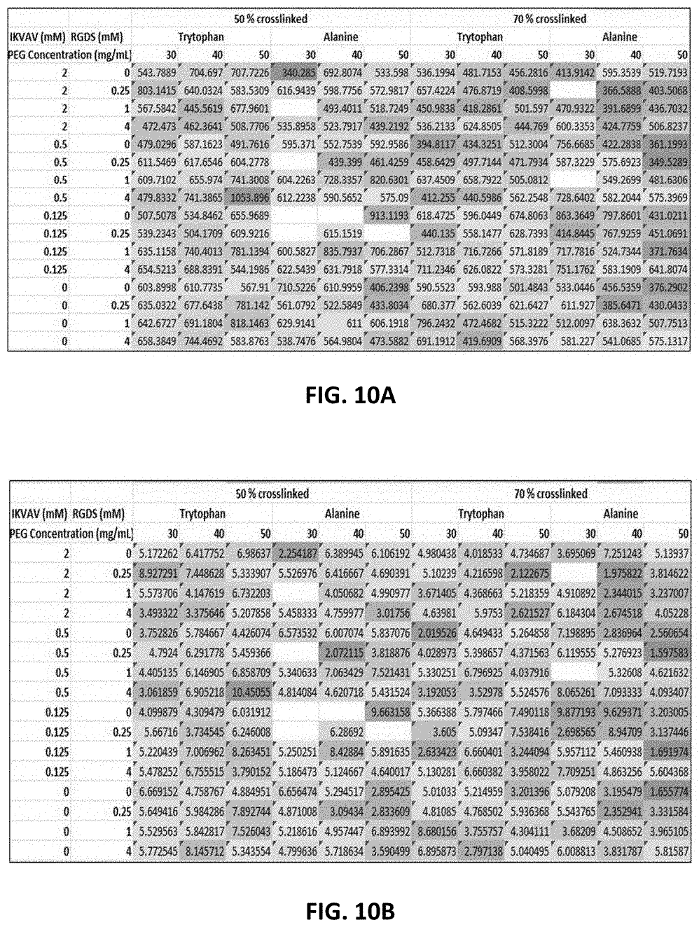

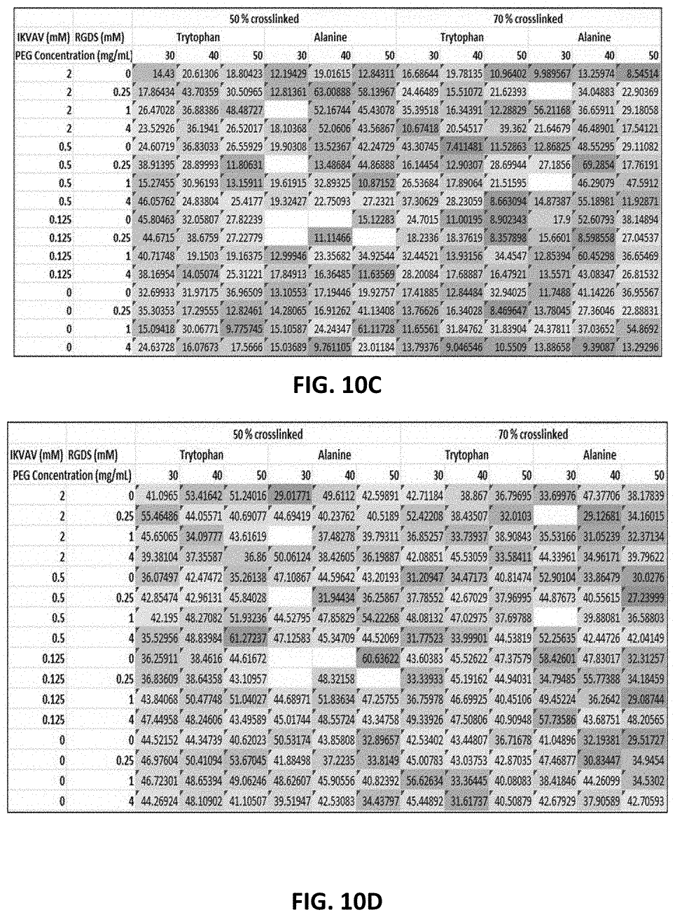

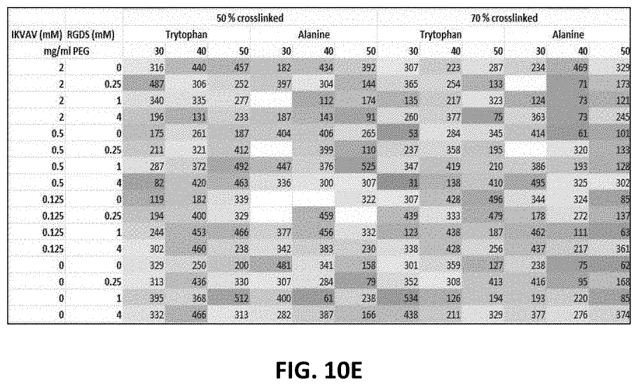

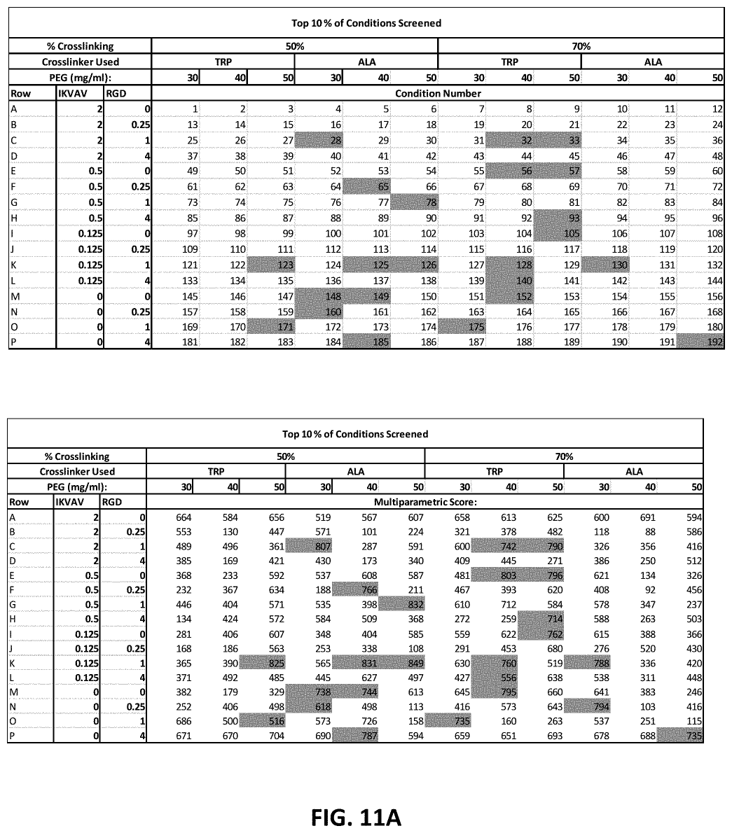

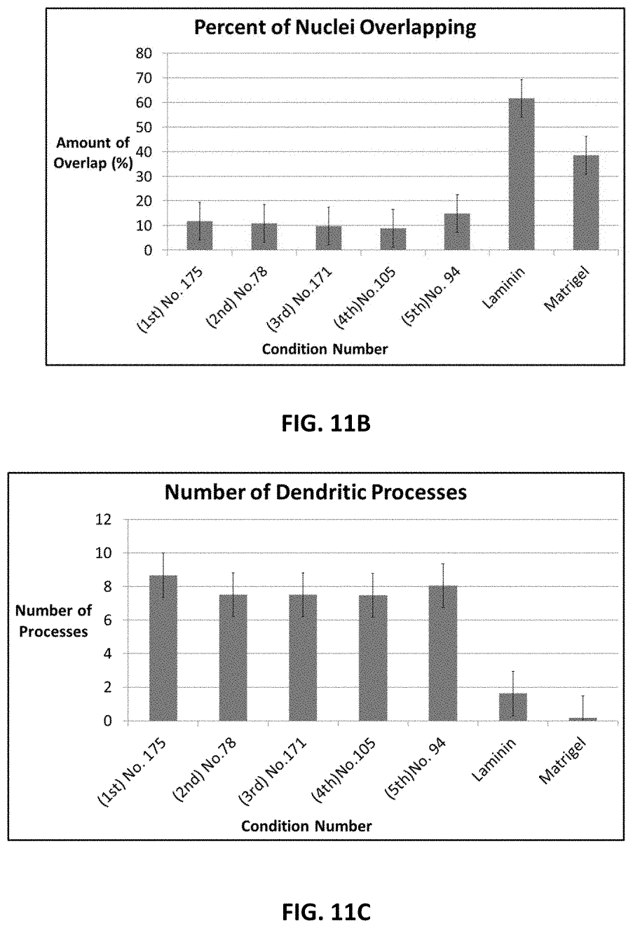

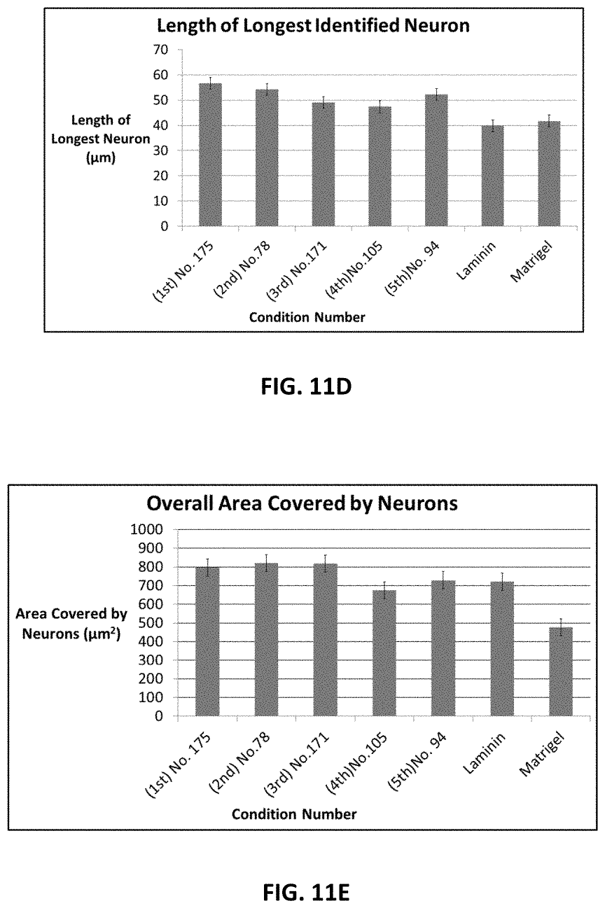

Using the results from each parameter of the high throughput software, the data was reorganized and then correlated to heat maps (FIGS. 10A-10E), which stratifies the data by 15% tiers. Each color on the map correlates to a 15% tier with the highest values indicated by green and the lowest values indicated in red. The gaps in the heat maps are primarily through outliers found in the initial screen. If a value was beyond or below 200% of the average value in the map, it was deemed an outlier and not counted in the analysis. A total of 8 conditions were considered outliers in this Example and subsequently not utilized in the heat map analysis.

Further, a comparison of conditions screened was made against the gold standard controls, laminin or MATRIGEL®. As shown in FIG. 10B, conditions identified showed less clustering of individual neurons than laminin or MATRIGEL®, allowing for easier high content imaging and assessment of individual cellular effects on neurons. As shown in FIG. 10B, there was an increase in the number of dendritic processes versus that of laminin or MATRIGEL®. Further, as shown in FIG. 10D, the hydrogels of the present disclosure supported longer neural outgrowth as compared to MATRIGEL® controls in the first 24 hours and larger cell somas than MATRIGEL® (FIG. 10E), indicating greater adhesion and cell spreading on hydrogel surfaces than that of MATRIGEL®.

These results demonstrate that the method for preparing hydrogel arrays as described herein provides the capability to control stiffness, immobilized ligand identity and ligand concentration (density), and soluble growth factor presentation. The hydrogel arrays of the present disclosure can support cell adhesion and survival and allow for screening complex cell-environment interactions.

### Example 2

In this Example, the hydrogel array of Example 1 was used to determine its effectiveness on neural screening. Particularly, this Example shows the ability to use the hydrogel array of Example 1 as a substrate for neural cell culture and screening of toxicity.

### Materials and Methods

Human iPSC-Derived Neuron Culture and Maintenance

Commercially available human iPSC-derived neurons (iCell Neurons) and their supporting media were purchased from Cellular Dynamics International (CDI, Madison, Wis.). Previously characterized iCell Neurons represent a mixture of post-mitotic GABAergic and glutamatergic neurons with >95% purity. The cells were received, frozen, thawed, and plated following a protocol recommended by CDI. Briefly, cells were plated on poly-L-lysine pre-coated 384 well plates (Greiner Bio-One) with PEG-NB hydrogels, 3.3 μg/ml laminin, or 60-120 μg/ml MATRIGEL®.

### Human NPC Culture and Maintenance

WTC11 NPCs (differentiated from WTC11 iPSC) at passage 5 were thawed in a 1×PBS solution containing 10% FBS (Gibco, Thermo Fisher). The NPCs were centrifuged at 300×g for 5 minutes, resuspended in N2B27+fibroblast growth factor (fgf) (R&D Systems), and mixed to singularize. Tissue-culture polystyrene plates were coated using MATRIGEL® at a density of 0.0087 μg cm⁻². The NPCs were seeded onto the plates and cultured for 24 hours at 37° C. in a 5% CO₂ atmosphere. The media was changed every other day under routine maintenance.

For passaging, the cells were incubated in Accutase (Corning) for 9-10 minutes at 37° C. until a majority of the cells were lifted. To dilute the Accutase, 4 ml of N2B27 medium per 1 ml Accutase was added to Accutase-treated cells. Afterwards, the cells were collected, centrifuged at 300×g for 5 minutes, resuspended in N2B27+fgf and mixed to singularize. Cells were seeded at 83×10³ cells cm⁻² onto fresh MATRIGEL®-coated plates.

### Preparation of PEG Solutions for Scaffold Optimization

The hydrogels used for examining iCell neuron network formation consisted of PEG-NB molecules, linear H-Cys-Arg-Gly-Asp-Gly-Ser-NH₂ (linear RGD) (SEQ ID NO: 47), linear H-Cys-Ile-Lys-Val-Ala-Val-NH₂ (linear IKVAV) (SEQ ID NO: 48), MMP-degradable H-Lys-Cys-Gly-Gly-Pro-Gln-Gly-Ile-Trp-Gly-Gln-Gly-Cys-Lys-NH₂ (SEQ ID NO: 49) crosslinking peptide (Genscript), and 0.1% w/v 12959 photoinitiator dissolved in phosphate buffered saline (1×PBS). The PEG-NB, adhesion peptides, and MMP-degradable crosslinker concentrations were adjusted to achieve varying levels of mechanical stiffness and adhesion capabilities in each system. Prior to the introduction of peptides in precursor solutions, the concentrations were verified using the Ellman's Assay (Thermo Fisher). To modulate neuron binding to the hydrogels, RGD and IKVAV molecules were added to solutions to achieve adhesion peptide concentrations between 0 and 4 mM and 0 and 2 mM, respectively. To vary the mechanical stiffness of the hydrogels, the PEG-NB backbone molecule was added to precursor solutions to achieve final concentrations between 30 and 50 mg/ml, and the MMP-degradable crosslinker volumes were altered to obtain final crosslinking percentages of 50 and 70%.

### Results

### Approach for the Identification of Synthetic Hydrogel Formulations.

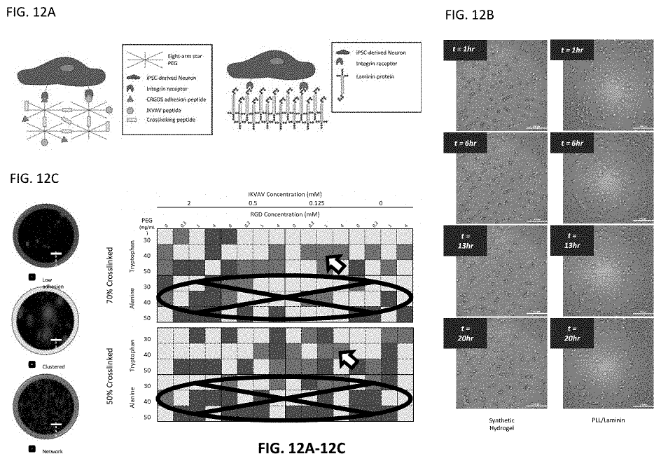

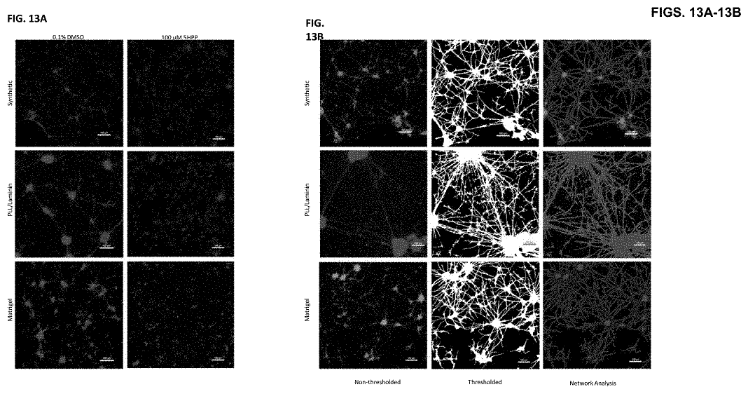

The approach for the identification of synthetic hydrogel formulations that supported the intended cellular behaviors (for example, adhesion and neurite extension) used arrays of thin hydrogels in a 384-well plate format that mimicked the essential properties of the native ECM. The addition of the pendant linear H-Cys-Arg-Gly-Asp-Ser-NH₂ (linear RGD) (SEQ ID NO: 47) peptide functions to mediate cell adhesion to the synthetic hydrogel through the RGD motif commonly found in integrin-binding ECM proteins. Mechanical properties and behaviors of the hydrogels were controlled by tuning the initial concentrations of 20 kDa, eight-arm PEG-norbornene (PEG-NB) and dithiol-terminated crosslinking molecules used to form the hydrogels (FIG. 12C). All hydrogel formulations were polymerized using the crosslinking peptide H-Lys-Cys-Gly-Gly-Pro-Gln-Gly-Ile-Trp-Gly-Gln-Gly-Cys-Lys-NH₂ (SEQ ID NO: 49) or H-Lys-Cys-Gly-Gly-Pro-Gln-Gly-Ile-Ala-Gly-Gln-Gly-Cys-Lys-NH₂ (SEQ ID NO: 50) that was degradable by matrix metalloproteinases (MMPs), thus enabling cell motility through cell-mediated degradation and remodeling of the hydrogel substrates.

Identification of materials to promote neural network formation. Hydrogel arrays formed in 384-well plates highlighted cell culture environments that supported complex neuronal network formation by iCell neurons.

### Utility of Synthetic Hydrogel for Network Inhibition Assays

Synthetic hydrogel-based network formation assays demonstrated an accurate and reproducible distinction between networks disrupted by the known microtubule-inhibitor colchicine and non-inhibited networks. Total network area was quantified by thresholding neurite processes and cell bodies stained with immunofluorescent tags for β_{III}-tubulin and MAP2. The resulting data was compared using the Z′ statistic to assess the accuracy of the synthetic hydrogel, PLL/Laminin, and MATRIGEL® screening systems in distinguishing neuronal networks disrupted by the known microtubule inhibitor Colchicine from non-inhibited networks.

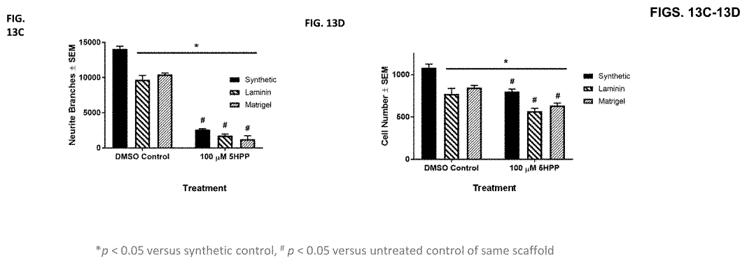

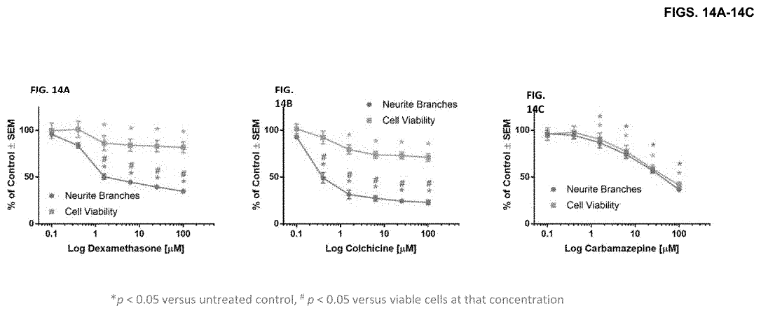

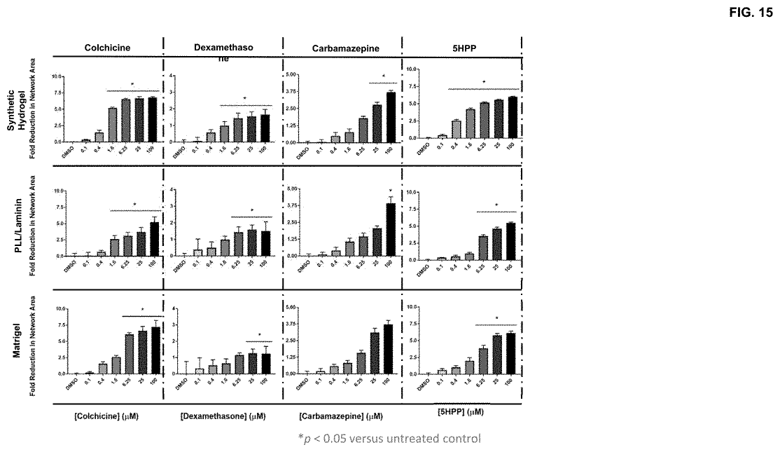

iCell neurons on synthetic hydrogels showed improved cellular characteristics compared with iCell neurons grown on PLL/Laminin and MATRIGEL® substrates, including total neurite branches and singularized cell number (FIGS. 13A-13D). Following quantification of the total number of neurite branches and total number of singularized cells, the synthetic hydrogels supported a significantly higher number of each parameter under the untreated, DMSO control conditions. Treatment of iCell neurons to 100 μM 5HPP demonstrated that significant reduction in each of these values continued to occur across all scaffolds, thus no scaffold acted in a manner which protected the iCell neurons from the inhibitory effects of each of the treatments. Cell clusters, as determined by a circular object greater than 40 μM in diameter, represented poor cellular behavior on a given scaffold. iCell neuron clustering on both PLL/Laminin and MATRIGEL® scaffolds was significantly greater compared to clustering on the synthetic hydrogel scaffold, in particular when comparing directly between PLL/Laminin and the hydrogels.

### Identification of Compounds Effecting Neuronal Networks Via Specific Inhibition

High-content imaging followed by skeleton network analysis identified compounds which perturbed neuronal networks through specific inhibition of neurite branches (FIGS. 14A-14C). iCell neurons grown on synthetic substrates showed significant reductions in neurite branches compared to the measured cell viability parameter when treated with both Dexamethasone (FIG. 14A) and Colchicine (FIG. 14B). Treatment of iCell neurons with Carbamazepine demonstrated apparent inhibition of neurite growth which is a secondary consequence of reduced viability, suggesting that inhibition is unspecific for this compound (FIG. 14C).

iPSC-Derived Neuron Sensitivity to Known Developmental Neurotoxicants

iCell Neuron networks on synthetic hydrogels showed increased sensitivity to known developmental neurotoxicants compared to PLL/Laminin and MATRIGEL® substrates.

What is claimed is:

1. A hydrogel composition for promoting neural cellular expansion and/or differentiation comprising: from about 20 mg/mL to about 100 mg/mL of a polyethylene glycol, at least about 0.125 mM cell adhesion peptide, and a soluble factor binder, and wherein the hydrogel composition has a degree of crosslinking ranging from about 50% to about 70%.

2. The hydrogel composition as set forth in claim 1 comprising from about 30 mg/mL to about 50 mg/mL polyethylene glycol.

3. The hydrogel composition as set forth in claim 1, wherein the polyethylene glycol is a polyethylene glycol functionalized with norbornene.

4. The hydrogel composition as set forth in claim 1 comprising from about 1 mM to about 4 mM cell adhesion peptide.

5. The hydrogel composition as set forth in claim 1, wherein the cell adhesion peptide is selected from the group consisting of RGDS (SEQ ID NO:1), CRGDS (SEQ ID NO:2), Acetylated-GCYGRGDSPG (SEQ ID NO:31); cyclic {RGD(Fd)C} (SEQ ID NO:33); CRGD-(G)13-PHSRN (SEQ ID NO:29); IKVAV (SEQ ID NO:37), and CPHSRN-(SG)5-RGD (SEQ ID NO:30).

6. The hydrogel composition as set forth in claim 1, wherein the cell adhesion peptide is selected from the group consisting of RGDS (SEQ ID NO:1) and IKVAV (SEQ ID NO:37).

7. The hydrogel composition as set forth in claim 6 comprising from about 0.125 mM to about 2 mM IKVAV (SEQ ID NO:37).

8. The hydrogel composition as set forth in claim 6 comprising from about 0.25 mM to about 4 mM RGDS (SEQ ID NO:1).

9. The hydrogel composition as set forth in claim 1 comprising a degree of alanine crosslinking of from about 50% to about 70%.

10. The hydrogel composition as set forth in claim 1 comprising a degree of trytophane crosslinking of from about 50% to about 70%.

11. A method of promoting cellular expansion, the method comprising:
preparing a hydrogel composition, wherein the hydrogel composition comprises from about 20 mg/mL to about 100 mg/mL of a polyethylene glycol, at least about 0.125 mM cell adhesion peptide, and a soluble factor binder, and wherein the hydrogel composition has a degree of crosslinking ranging from about 50% to about 70%;
contacting a cell with the hydrogel composition; and
culturing the cell.

12. The method as set forth in claim 11, wherein the cell is selected from the group consisting of an embryonic stem cell-derived neuron, an embryonic stem cell-derived neural progenitor cell, an embryonic stem cell-derived astrocyte, an embryonic stem cell-derived microglial cell, an induced pluripotent stem cell-derived neural progenitor cell, an induced pluripotent stem cell-derived astrocyte, an induced pluripotent stem cell-derived microglial cell, a neuron, and combinations thereof.

13. The method as set forth in claim 11, wherein the polyethylene glycol is a polyethylene glycol functionalized with norbornene.

14. The method as set forth in claim 11 comprising from about 1 mM to about 4 mM cell adhesion peptide.

15. The method as set forth in claim 11, wherein the cell adhesion peptide is selected from the group consisting of RGDS (SEQ ID NO:1), CRGDS (SEQ ID NO:2), Acetylated-GCYGRGDSPG (SEQ ID NO:31); cyclic {RGD(Fd)C} (SEQ ID NO:33); CRGD-(G)13-PHSRN (SEQ ID NO:29); IKVAV (SEQ ID NO:37), and CPHSRN-(SG)5-RGD (SEQ ID NO:30).

16. The method as set forth in claim 11, wherein the cell adhesion peptide is selected from the group consisting of RGDS (SEQ ID NO:1) and IKVAV (SEQ ID NO:37).

17. The method as set forth in claim 16 comprising from about 0.125 mM to about 2 mM IKVAV (SEQ ID NO:37).

18. The method as set forth in claim 16 comprising from about 0.25 mM to about 4 mM RGDS (SEQ ID NO:1).

19. The method as set forth in claim 11 comprising a degree of alanine crosslinking of from about 50% to about 70%.

20. A method for neurotoxicity screening of cells, the method comprising:
preparing a hydrogel composition, wherein the hydrogel composition comprises from about 20 mg/mL to about 100 mg/mL of a polyethylene glycol, at least about 0.125 mM cell adhesion peptide, and a soluble factor binder, and wherein the hydrogel composition has a degree of crosslinking ranging from about 50% to about 70%;
contacting a cell with the hydrogel composition;
culturing the cell to form a network;
contacting the network with a candidate neurotoxicant; and
analyzing the growth of the network in the presence of the candidate neurotoxicant.

21. The method as set forth in claim 20, wherein the cell is selected from the group consisting of an embryonic stem cell-derived neuron, an embryonic stem cell-derived neural progenitor cell, an embryonic stem cell-derived astrocyte, an embryonic stem cell-derived microglial cell, an induced pluripotent stem cell-derived neural progenitor cell, an induced pluripotent stem cell-derived astrocyte, an induced pluripotent stem cell-derived microglial cell, a neuron, and combinations thereof.

22. The method as set forth in claim 20, wherein the polyethylene glycol is a polyethylene glycol functionalized with norbornene.

23. The method as set forth in claim 20, wherein the cell adhesion peptide is selected from the group consisting of RGDS (SEQ ID NO:1), CRGDS (SEQ ID NO:2), Acetylated-GCYGRGDSPG (SEQ ID NO:31); cyclic {RGD(Fd)C} (SEQ ID NO:33); CRGD-(G)13-PHSRN (SEQ ID NO:29); IKVAV (SEQ ID NO:37), and CPHSRN-(SG)5-RGD (SEQ ID NO:30).

24. The method as set forth in claim 20 comprising a degree of alanine crosslinking of from about 50% to about 70%.

25. The method as set forth in claim 20 comprising a degree of trytophane crosslinking of from about 50% to about 70%.
